(19) United States
(12) Patent Application Publication
(10) Pub. No.: US 2016/0367395 A1
(43) Pub. Date: Dec. 22, 2016

(54) BODY TEMPERATURE ADJUSTMENT SHEET

(71) Applicant: MUYU CO., LTD., Tokyo (JP)

(72) Inventor: Hiroshi ITO, Tokyo (JP)

(73) Assignee: MUYU CO., LTD., Tokyo (JP)

(21) Appl. No.: 15/255,777

(22) Filed: Sep. 2, 2016

Related U.S. Application Data
(63) Continuation of application No. PCT/JP2014/065070, filed on Jun. 6, 2014.

(51) Int. Cl. A61F 7/03; A61F 7/10

(52) U.S. Cl. CPC A61F 7/03; A61F 7/10; A61F 2007/0031

(57) ABSTRACT

Provided is a body temperature adjustment sheet having a structure suitable for cooling or warming the underarm. A body temperature adjustment sheet is a cooling sheet for cooling the underarm of a person. The body temperature adjustment sheet has an opening and has an annular shape. The opening is an opening through which the arm of a person is passed. The body temperature adjustment sheet is fixed to the underarm with the arm passing through the opening and cools the underarm.

### CROSS REFERENCE TO RELATED APPLICATION

This is a Continuation of International Application No. PCT/JP20141065070 filed Jun. 6, 2014. The contents of this application are hereby incorporated by reference in their entirety.

### TECHNICAL FIELD

The present invention relates to a body temperature adjustment sheet that cools or warms the body of a person.

### BACKGROUND ART

Patent Document 1 discloses a cooling sheet, which is a kind of body temperature adjustment sheet. The cooling sheet disclosed in the document includes a water-containing gel layer, and is used by being attached to the body of a person when the person has a fever, for example. The cooling sheet is configured to cool the body with latent heat of vaporization of water contained in the water-containing gel layer.

### CITATION LIST

### Patent Document

Patent Document 1: JP 2006-95200A

### SUMMARY OF INVENTION

### Technical Problem

Lymph nodes are concentrated in the underarm of a person. For this reason, the body temperature can be adjusted efficiently by cooling or warming the underarm. However, the underarm is an area where heavy sweating occurs and active movement of the skin takes place. Accordingly, even if the cooling sheet described above is attached to the underarm, it is likely to separate from the underarm, and it is therefore difficult to fix the cooling sheet to the underarm for a long period of time. Thus, the conventional body temperature adjustment sheet is not suitable for cooling or warming the underarm.

### Solution to Problem

The present invention has been made in view of the problem described above, and it is an object of the present invention to provide a body temperature adjustment sheet having a structure suitable for cooling or warming the underarm.

A body temperature adjustment sheet according to the present invention is an annular body temperature adjustment sheet having an opening through which an arm of a person is passed, wherein the body temperature adjustment sheet is fixed to an underarm with the arm passing through the opening, and cools or warms the underarm.

The body temperature adjustment sheet is configured to cool or warm the underarm of a person, and it is therefore possible to efficiently adjust the body temperature. Furthermore, the body temperature adjustment sheet has an opening, and is fixed to the underarm with the arm passing through the opening. For this reason, the body temperature adjustment sheet is likely to be fixed to the underarm for a long period of time.

### Advantageous Effects of Invention

According to the present invention, it is possible to implement a body temperature adjustment sheet having a structure suitable for cooling or warming the underarm.

### BRIEF DESCRIPTION OF DRAWINGS

FIG. 1 is a plan view showing a body temperature adjustment sheet according to a first embodiment of the present invention,

FIG. 2 is a diagram illustrating the body temperature adjustment sheet shown in FIG, 1 fixed to the underarm,

FIG. 3 is a cross-sectional view of the body temperature adjustment sheet taken along the line III-III shown in FIG. 1.

FIG. 4 is a diagram illustrating the effects of the body temperature adjustment sheet shown in FIG. 1.

FIG. 5 is a plan view showing a body temperature adjustment sheet according to a second embodiment of the present invention.

FIG. 6 is a plan view showing a body temperature adjustment sheet according to a third embodiment of the present invention.

FIG. 7 is a cross sectional view of the body temperature adjustment sheet taken along the line VII-VII shown in FIG. 6.

FIG. 8 is a cross-sectional view of a body temperature adjustment sheet according to a variation.

### DESCRIPTION OF EMBODIMENTS

Hereinafter, embodiments of the present invention will be described in detail with reference to the drawings. In the description of the drawings, the same reference numerals are given to the same components, and a redundant description will be omitted.

### First Embodiment

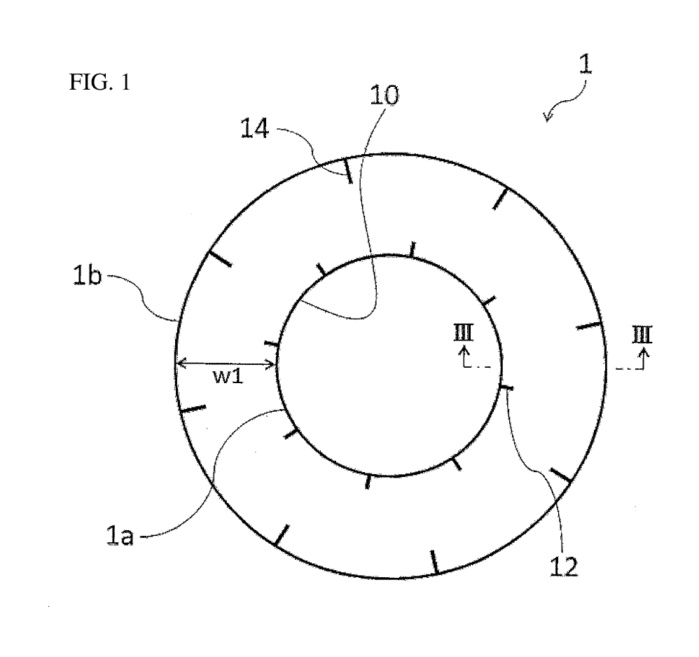

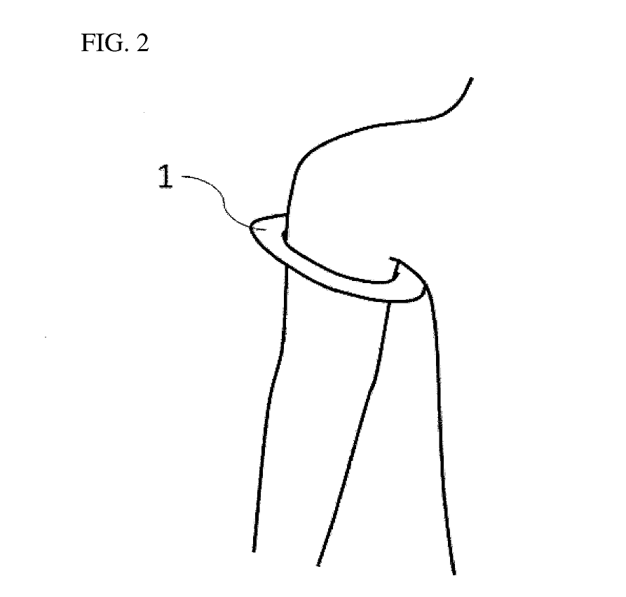

FIG. 1 is a plan view showing a body temperature adjustment sheet according to a first embodiment of the present invention. A body temperature adjustment sheet 1 is a cooling sheet for cooling the underarm of a person. The body temperature adjustment sheet 1 has an opening 10 and has an annular shape. The opening 10 is an opening through which the arm of a person is passed, and is formed in a face of the body temperature adjustment sheet 1. As shown in FIG. 2, the body temperature adjustment sheet 1 is fixed to the underarm with the arm passing through the opening 10 and cools the underarm.

In the present embodiment, the body temperature adjustment sheet 1 has an inner circumference (the edge of the opening 10) 1a and an outer circumference 1b that are circular in shape. The center of the inner circumference 1a and the center of the outer circumference 1b are coincident. To be specific, a circle constituting the inner circumference la and a circle constituting the outer circumference 1b are concentric circles. Accordingly, the body temperature adjustment sheet 1 has a constant width w1 along the circumferential direction. The width w1 is, for example, about 4 to 8 cm. The opening 10 has a diameter of, for example, about 8 to 12 cm.

In the body temperature adjustment sheet 1, a slit portion 12 (first slit portion) and a slit portion 14 (second slit portion) are formed. The slit portion 12 extends outward from the inner circumference 1a. In the present embodiment, a plurality of (namely, eight) slit portions 12 are formed. These slit portions 12 extend radially from the opening 10. In addition, the slit portion 14 extends inward from the outer circumference 1b. In the present embodiment, a plurality of (namely, eight) slit portions 14 are formed. These slit portions 14 extend radially about the opening 10. Each of the slit portions 12 and 14 has a length of, for example, about 0.5 to 1.5 cm.

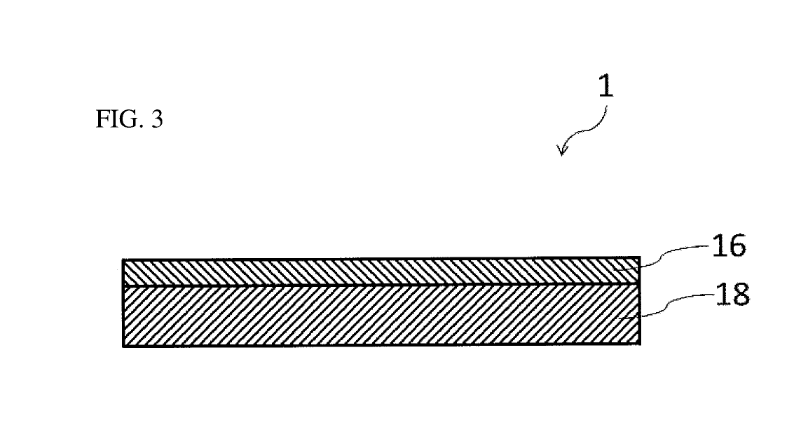

FIG. 3 is a cross-sectional view taken along the line shown in FIG. 1. The body temperature adjustment sheet 1 has a stacked structure composed of a support sheet 16 and a water-containing gel layer 18. The support sheet 16 has moisture permeability and air permeability. As the material of the support sheet 16, for example, a non-woven fabric can be used.

In the present embodiment, the water-containing gel layer 18 is provided on approximately the entire back surface of the support sheet 16. The water-containing gel layer 18 cools the underarm while it is in contact with the underarm. To be specific, the underarm is cooled with latent heat of vaporization of water contained in the water-containing gel layer 18. As the material of the water-containing gel layer 18, it is possible to use, for example, a natural polymer such as dextrin, xanthan gum, sodium alginate, mannan, gelatin or pectin; or a synthetic polymer such as polyvinyl alcohol (PVA), sodium polyacrylate or carboxymethyl cellulose (CMC).

The body temperature adjustment sheet 1 may contain an antimicrobial component and/or a fragrance component. Examples of the antimicrobial component include tannin such as catechin. Tannins contained in tea or coffee can be used as the tannin. Examples of the fragrant component include a component that releases a herbal fragrance such as eucalypt and rosemary. These components may be contained in both the support sheet 16 and the water-containing gel layer 18, or may be contained in only one of them.

The effects of the body temperature adjustment sheet 1 will be described. The body temperature adjustment sheet 1 is configured to cool the underarm of a person, and it is therefore possible to efficiently adjust the body temperature. Furthermore, the body temperature adjustment sheet 1 has the opening 10, and is fixed to the underarm with the arm passing through the opening 10. For this reason, the body temperature adjustment sheet 1 is likely to be fixed to the underarm for a long period of time. Accordingly, a body temperature adjustment sheet 1 having a structure suitable for cooling the underarm is implemented.

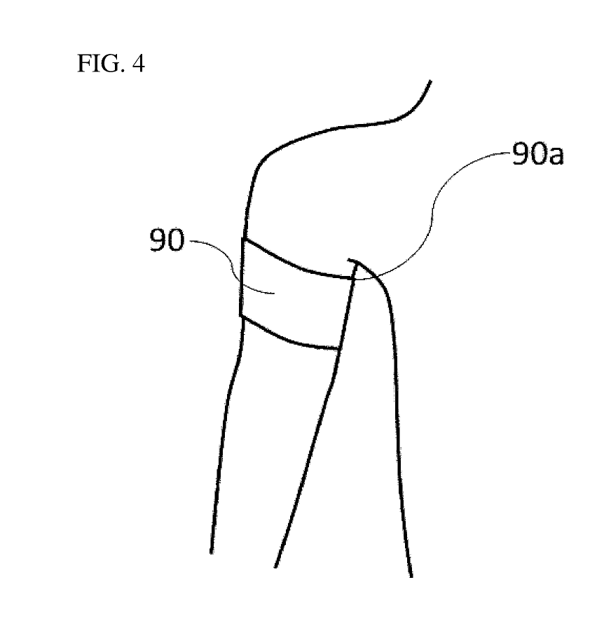

As a means for fixing the body temperature adjustment sheet to the underarm, as shown in FIG. 4, a band-shaped body temperature adjustment sheet 90 may be wound around the upper arm portion. However, in this case, the surface of the body temperature adjustment sheet 90 is parallel to the direction of extension of the arm. For this reason, a portion of the body temperature adjustment sheet 90 that is in contact with the underarm is limited to a small area near an upper end 90a of the body temperature adjustment sheet 90.

In contrast, in the case of the present embodiment, the opening 10 is formed in the face of the body temperature adjustment sheet 1, and thus the surface of the body temperature adjustment sheet 1 is perpendicular to the direction of extension of the arm (the direction of the central axis of the opening 10) (see FIG. 2). To be specific, the surface of the body temperature adjustment sheet 1 has a positional relation of opposing the underarm. Accordingly, the body temperature adjustment sheet 1 (the water-containing gel layer 18) can be brought into contact with the underarm over a wide range. It goes without saying that the cooling effect can be enhanced by increasing the contact area between the body temperature adjustment sheet 1 and the underarm as described above.

In the body temperature adjustment sheet 1, the slit portions 12 are formed. Accordingly, the arm can be easily passed through the opening 10 when attaching the body temperature adjustment sheet 1 to the arm. In the present embodiment, the plurality of slit portions 12 are formed radially, and are thus particularly helpful in allowing the arm to pass through the opening.

In the body temperature adjustment sheet 1, the slit portions 14 are also formed. Accordingly, the body temperature adjustment sheet 1 can easily deform, and thus the degree of adhesion of the body temperature adjustment sheet 1 to the underarm can be enhanced. In the present embodiment, the plurality of slit portions 14 are formed radially, and thus are particularly helpful in enhancing the degree of adhesion to the underarm.

The body temperature adjustment sheet 1 has a stacked structure composed of the support sheet 16 and the water-containing gel layer 18. By providing the support sheet 16 in this way, the entire shape of the body temperature adjustment sheet 1 can be easily retained, and at the same time, the stickiness of the front surface of the body temperature adjustment sheet 1 (the surface being opposite to the surface that is brought into contact with the underarm) can be suppressed. Also, by providing the water-containing gel layer 18, the underarm can be cooled with a simple configuration. Furthermore, the adhesive force of the water-containing gel layer 18, together with fixing by the opening 10, can make the body temperature adjustment sheet 1 fix more reliably to the underarm.

In the case where the body temperature adjustment sheet 1 contains an antimicrobial component, it is possible to suppress the propagation of bacteria contained in the underarm sweat and the like. Also, in the case where the body temperature adjustment sheet 1 contains a fragrance component, it is possible to mask odors such as the odor of underarm sweat.

### Second Embodiment

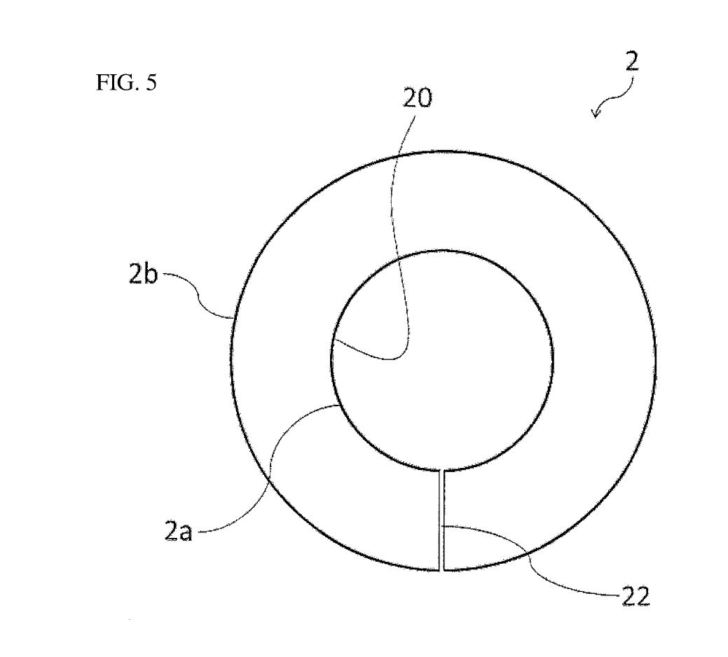

FIG. 5 is a plan view showing a body temperature adjustment sheet according to a second embodiment of the present invention. A body temperature adjustment sheet 2 is a cooling sheet for cooling the underarm of a person. The body temperature adjustment sheet 2 has an opening 20 and has an annular shape. The opening 20 is an opening through which the arm of a person is passed, and is formed in a face of the body temperature adjustment sheet 2. The body temperature adjustment sheet 2 is fixed to the underarm with the arm passing through the opening 20, and cools the underarm.

The body temperature adjustment sheet 2 has an inner circumference 2a and an outer circumference 2b that are circular in shape. In addition, the center of the inner circumference 2a and the center of the outer circumference 2b are coincident. In the body temperature adjustment sheet 2, a cut portion 22 is formed. The cut portion 22 extends from the inner circumference 2a to the outer circumference 2b of the body temperature adjustment sheet 2, and divides a portion of the body temperature adjustment sheet 2. The body temperature adjustment sheet 2 has the same stacked structure as that of the body temperature adjustment sheet 1 (see FIG. 3).

The effects of the body temperature adjustment sheet 2 will be described. The body temperature adjustment sheet 2 is configured to cool the underarm of a person, and it is therefore possible to efficiently adjust the body temperature. Furthermore, the body temperature adjustment sheet 2 has the opening 20, and is fixed to the underarm with the arm passing through the opening 20. For this reason, the body temperature adjustment sheet 2 is likely to be fixed to the underarm for a long period of time. Accordingly, a body temperature adjustment sheet 2 having a structure suitable for cooling the underarm is implemented.

In the body temperature adjustment sheet 2, the cut portion 22 is formed. The cut portion 22 divides a portion of the body temperature adjustment sheet 2, and thus the body temperature adjustment sheet 2 can be more easily attached to the arm. To be specific, in the case of a configuration in which the cut portion 22 is not provided, in order to attach the body temperature adjustment sheet 2 to the arm, the hand, the forearm portion and the upper arm portion need to be passed in sequence through the opening 20. However, in the case of the configuration in which the cut portion 22 is provided, the upper arm portion can be passed through the opening 20 directly from the cut portion 22. The other effects of the body temperature adjustment sheet 2 are the same as those described in connection with the body temperature adjustment sheet 1.

### Third Embodiment

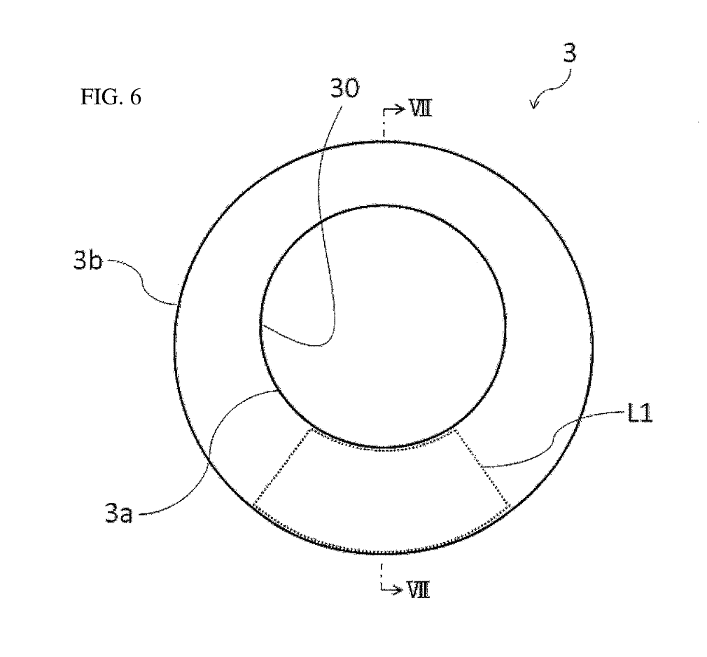

FIG. 6 is a plan view showing a body temperature adjustment sheet according to a third embodiment of the present invention. A body temperature adjustment sheet 3 is a cooling sheet for cooling the underarm of a person. The body temperature adjustment sheet 3 has an opening 30 and has an annular shape. The opening 30 is an opening through which the arm of a person is passed, and is formed in a face of the body temperature adjustment sheet 3. The body temperature adjustment sheet 3 is fixed to the underarm with the arm passing through the opening 30 and cools the underarm.

The body temperature adjustment sheet 3 has an inner circumference 3a and an outer circumference 3b that are circular in shape. Note that the center of the inner circumference 3a and the center of the outer circumference 3b are not coincident. Accordingly, the body temperature adjustment sheet 3 on one side of the opening 30 (the lower side in FIG. 6) has a greater width than the width of the body temperature adjustment sheet 3 on the other side of the opening 30 (the upper side in FIG. 6).

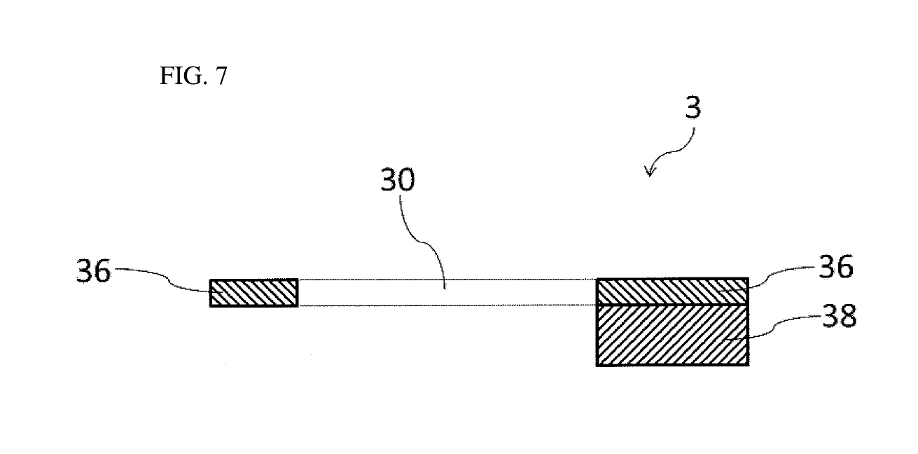

FIG. 7 is a cross-sectional view taken along the line VII-VII shown in FIG. 6. The body temperature adjustment sheet 3 has a stacked structure composed of a support sheet 36 and a water-containing gel layer 38. The support sheet 36 has moisture permeability and air permeability, as with the support sheet 16. In the present embodiment, the water-containing gel layer 38 is provided only on a portion of the back surface of the support sheet 36 (the portion surrounded by the dotted line L1 in FIG. 6). To be specific, the water-containing gel layer 38 is provided only on one side of the opening 30 (on the side where the body temperature adjustment sheet 3 has the greater width). The water-containing gel layer 38 cools the underarm while it is in contact with the underarm, as with the water-containing gel layer 18.

The effects of the body temperature adjustment sheet 3 will be described. The body temperature adjustment sheet 3 is configured to cool the underarm of a person, and it is therefore possible to efficiently adjust the body temperature. Furthermore, the body temperature adjustment sheet 3 has the opening 30, and is fixed to the underarm with the arm passing through the opening 30. For this reason, the body temperature adjustment sheet 3 is likely to be fixed to the underarm for a long period of time. Accordingly, a body temperature adjustment sheet 3 having a structure suitable for cooling the underarm is implemented.

In the body temperature adjustment sheet 3, the center of the inner circumference 3a and the center of the outer circumference 3b are not coincident, and the body temperature adjustment sheet 3 on one side of the opening 30 has a greater width than the width of the body temperature adjustment sheet 3 on the other side of the opening 30. Accordingly, when the portion where the body temperature adjustment sheet 3 has the greater width is brought into contact with the underarm, the portion where the body temperature adjustment sheet 3 has the smaller width is positioned on the outer side of the upper arm portion. Accordingly, even if the user wears a sleeved garment with the body temperature adjustment sheet 3 being attached to his/her arm, the body temperature adjustment sheet 3 is unlikely to interrupt the action of the user.

The water-containing gel layer 38 is provided only on a portion of the support sheet 36. By partially providing the water-containing gel layer 38 in this way, the production cost of the body temperature adjustment sheet 3 can be reduced. The other effects of the body temperature adjustment sheet 3 are the same as those described in connection with the body temperature adjustment sheet 1.

The body temperature adjustment sheet according to the present invention is not limited to the embodiments given above, and various modifications can be made. In the embodiments given above, examples are shown in which the inner circumference and the outer circumference of the body temperature adjustment sheet are circular in shape. However, the inner circumference and the outer circumference of the body temperature adjustment sheet may be elliptic or any other shape.

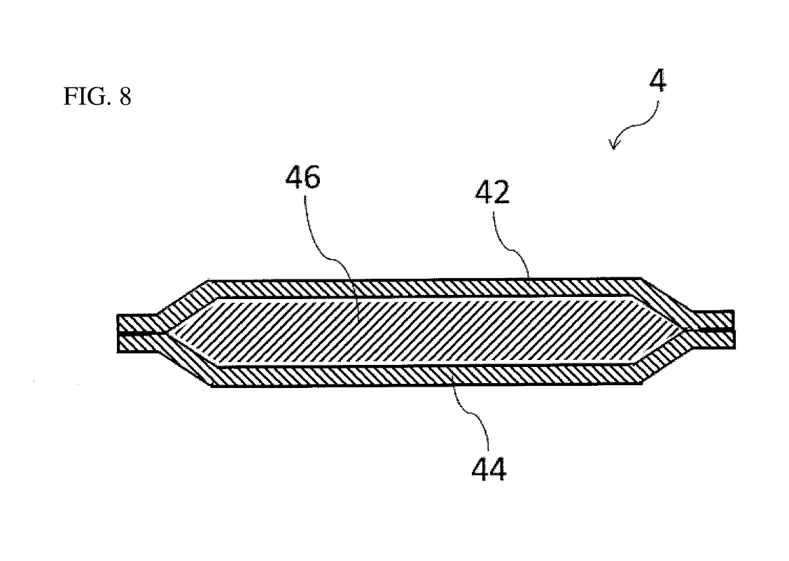

In the embodiments given above, examples are shown in which the body temperature adjustment sheet is a cooling sheet. However, the body temperature adjustment sheet may be a warming sheet for warming the underarm of a person. FIG. 8 is a cross-sectional view showing an example of such a body temperature adjustment sheet. A body temperature adjustment sheet 4 includes a front sheet 42, a back sheet 44, and a heat generation portion 46. The front sheet 42 and the back sheet 44 are bonded to each other at their peripheral edge portions, thereby forming a bag. The front sheet 42 and the back sheet 44 have moisture permeability and air permeability. As the material of the front sheet 42 and the back sheet 44, for example, a non-woven fabric can be used.

The heat generation portion 46 is provided in the bag formed by the front sheet 42 and the back sheet 44. The heat generation portion 46 may be configured to generate heat together with the generation of water vapor, or, like a disposable heating pad, may be configured to generate heat without the generation of water vapor. The heat generation portion 46 is made of, for example, a heat generator containing an oxidizable metal, activated carbon and water. The oxidizable metal is a metal that generates oxidation reaction heat. As the oxidizable metal, for example, iron, aluminum, zinc, manganese, magnesium or calcium can be used.

The body temperature adjustment sheet 4 has the same planar shape as the planar shape of the body temperature adjustment sheet 1 (FIG. 1), the body temperature adjustment sheet 2 (FIG. 5) or the body temperature adjustment sheet 3 (FIG. 6). To be specific, the body temperature adjustment sheet 4 also has an opening, and is fixed to the underarm with the arm passing through the opening. At this time, the body temperature adjustment sheet 4 warms the underarm while the back sheet 44 is in contact with the underarm, To be specific, the underarm is warmed by oxidation reaction heat generated in the heat generation portion 46. The heat generation portion 46 may be provided in approximately the entire body temperature adjustment sheet 4, as with the water-containing gel layer 18, or may be provided only on a portion of the body temperature adjustment sheet 4, as with the water-containing gel layer 38.

### LIST OF REFERENCE NUMERALS

1 Body temperature adjustment sheet

2 Body temperature adjustment sheet

3 Body temperature adjustment sheet

4 Body temperature adjustment sheet

10 Opening

12 Slit portion (First slit portion)

14 Slit portion (Second slit portion)

16 Support sheet

18 Water-containing gel layer

20 Opening

22 Cut portion

30 Opening

36 Support sheet

38 Water-containing gel layer

42 Front sheet

44 Back sheet

46 Heat generation portion

1. An annular body temperature adjustment sheet having an opening through which an arm of a person is passed,
wherein the body temperature adjustment sheet is fixed to an underarm with the arm passing through the opening, and cools or warms the underarm.

2. The body temperature adjustment sheet according to claim 1,
wherein the body temperature adjustment sheet includes a first slit portion that extends outward from an inner circumference of the body temperature adjustment sheet.

3. The body temperature adjustment sheet according to claim 2,
wherein a plurality of the first slit portions are formed, and
the plurality of first slit portions extend radially from the opening.

4. The body temperature adjustment sheet according to claim 1,
wherein the body temperature adjustment sheet includes a second slit portion that extends inward from an outer circumference of the body temperature adjustment sheet,

5. The body temperature adjustment sheet according to claim 4,
wherein a plurality of the second slit portions are formed, and
the plurality of second slit portions extend radially about the opening.

6. The body temperature adjustment sheet according to claim 1,
wherein the body temperature adjustment sheet includes a cut portion that extends from an inner circumference to an outer circumference of the body temperature adjustment sheet.

7. The body temperature adjustment sheet according to claim 1,
wherein the body temperature adjustment sheet on one side of the opening has a greater width than a width of the body temperature adjustment sheet on the other side of the opening.

8. The body temperature adjustment sheet according to claim 1,
wherein an inner circumference and an outer circumference of the body temperature adjustment sheet are circular or elliptic in shape.

9. The body temperature adjustment sheet according to claim 8,
wherein a center of the inner circumference and a center of the outer circumference are not coincident with each other.

10. The body temperature adjustment sheet according to claim 1,
wherein the body temperature adjustment sheet contains an antimicrobial component.

11. The body temperature adjustment sheet according to claim 10,
wherein the antimicrobial component is tannin.

12. The body temperature adjustment sheet according to claim 1,
wherein the body temperature adjustment sheet contains a fragrance component.

13. The body temperature adjustment sheet according to claim 12,
wherein the fragrance component releases a herbal fragrance.

14. The body temperature adjustment sheet according to claim 1,
wherein the body temperature adjustment sheet is configured to cool the underarm.

15. The body temperature adjustment sheet according to claim 14,
wherein the body temperature adjustment sheet has a stacked structure including a support sheet and a water-containing gel layer.

16. The body temperature adjustment sheet according to claim 1,
wherein the body temperature adjustment sheet is configured to warm the underarm.

17. The body temperature adjustment sheet according to claim 16, comprising:
a front sheet;
a back sheet that forms a bag together with the front sheet; and
a heat generation portion provided in the bag.
